(19) United States
(12) Patent Application Publication
(10) Pub. No.: US 2014/0276567 A1
(43) Pub. Date: Sep. 18, 2014

(54) Device and System for Dispensing a Biological Sealant

(71) Applicant: John Goodman, Ann Arbor, MI (US)

(72) Inventor: John Goodman, Ann Arbor, MI (US)

(21) Appl. No.: 13/836,058

(22) Filed: Mar. 15, 2013

(51) Int. Cl. A61M 5/19; A61B 17/00

(52) U.S. Cl. CPC A61M 5/19; A61B 17/00491
USPC 604/506; 606/214

(57) ABSTRACT

A system and method for delivery of a biological material using a gas-assisted delivery, including a gas entry port at the proximal end of the delivery system, particularly useful for the delivery of a two-part biological component.

### FIELD OF THE INVENTION

The present invention is directed to a system and method for dispensing a material. More specifically, the system and method is directed to dispensing a biological material, such as a biological sealant, and incorporates a gas assisted spray mechanism for delivery.

### BACKGROUND OF THE INVENTION

Applying a sealant to a target area has a number of benefits, particularly in the medical field, where such sealants may be used to treat biological tissue. The use of biological sealants can greatly aid in the treatment and recovery of an individual both during and after a medical procedure. Some biological sealants are absorbable by the body of an individual, and thus are extremely useful in treating biological tissue since the sealants do not need to be removed from the individual after treatment. Application of any sealants, including biological sealants, to target areas entails precision and accuracy. This is particularly true when the target area is the body of an individual and the sealant is intended to be used to seal or repair tissue. Such accuracy and precision is further complicated when a multi-part sealant is used, such as the two-part formation of fibrin, which requires mixing of thrombin and fibrinogen immediately prior to application. Delivery of such multi-part compositions can prove difficult, particularly due to the rapid polymerization upon interaction of the components. For delivery of fibrin, for example, the two components are typically dispensed simultaneously from separate devices, such as syringes, and mixed together immediately prior to application. Syringes for such delivery include those described in U.S. Pat. No. 5,814,022, the entire content of which is incorporated by reference herein.

In order to apply a material such as a sealant to the desired site, the material must be prepared, such as by placing multiple components into an apparatus, and then dispensing those components simultaneously. Pressurized application of sealant, such as through use of a syringe described above, is frequently used to deliver the sealant to the desired site, and sometimes pressurized and atomized application of the sealant is useful. For a multi-part combination sealant, accuracy of the application may be aided by use of gas-assisted pressure to spray the material directly at the intended site. Using a sealant that is atomized, such as through the use of a pressurized, inert or sterile gas, sprays the material in a pressurized manner through an opening in the apparatus.

Typical gas-assisted delivery devices, however, use gas hose connections that connect to the device at or near the dispensing tip. This configuration poses a number of complications and limitations on the system, including obstruction of part of the operator's view to the site, lack of proper balance to the system, and connection/disconnection issues. The present invention seeks to remedy these and other issues by providing a useful sealant delivery system that incorporates gas-assisted delivery.

### SUMMARY OF THE INVENTION

The present invention is related to a device for dispensing a multi-part composition and method of using the device. Various components may be present in the invention, including for example, a headpiece, an applicator, a dispenser, a loading cartridge, and other components as will be explained below. Systems and devices to load and dispense materials, including biological materials, are provided. Methods of use are also provided.

In one embodiment, there is provided a device for dispensing a biological sealant including: a headpiece having proximal and distal ends including: a first tubular barrel and second tubular barrel, each barrel disposed between the proximal and distal ends of the headpiece, each barrel including an exit port at the distal end of the headpiece; a plunger extending into the proximal end of each barrel; a pressurized gas conduit disposed between the proximal and distal ends of the headpiece, the gas conduit having a gas entry port at the proximal end of the headpiece and a gas exit port at the distal end of the headpiece; and an attachment mechanism located at the distal end of the headpiece; and an applicator having proximal and distal ends, including; a mating structure at the proximal end of the applicator, the mating structure configured to be releasably engaged with the attachment mechanism on the headpiece; a dispensing structure disposed at the distal end of the applicator; a first and second fluid passageway, each fluid passageway being in fluid communication with one of the exit ports; and a first gas passageway in fluid communication with the gas exit port; and an actuator located on the headpiece for controlling pressurized fluid flow through the pressurized gas conduit.

Other embodiments provide a method of delivering a biological material to a delivery site, including the steps of: providing a device for dispensing a biological material, the device including: a headpiece having proximal and distal ends including: a first tubular barrel and second tubular barrel, each barrel disposed between the proximal and distal ends of the headpiece for transporting sealant materials, each barrel including an exit port at the distal end of the headpiece; a plunger extending into each barrel at the proximal end; a pressurized gas conduit disposed between the proximal and distal ends of the headpiece for transporting pressurized fluid, the gas conduit having a gas exit port at the distal end of the headpiece; and an attachment mechanism located at the distal end of the headpiece; an applicator having proximal and distal ends, including: a mating structure at the proximal end of the applicator, the mating structure configured to be releasably engaged with the attachment mechanism on the headpiece; a dispensing structure disposed at the distal end of the applicator; a first and second fluid passageway, each fluid passageway being in fluid communication with one of the exit ports and terminating at the dispensing structure; a first gas passageway in fluid communication with the gas exit port; and an actuator located on the headpiece for controlling pressurized fluid flow through the pressurized gas conduit; at least partially filling the first barrel with a first biological material and at least partially filling the second barrel with a second biological material; moving each of the plungers in a distal direction so as to force the first biological material from the first barrel through the first fluid passageway and the second biological material from second barrel through the second fluid passageway; moving the actuator so as to permit the flow of pressurized gas to travel through the gas conduit through the first gas passageway; atomizing the biological material to form an atomized composition; and dispensing the atomized composition at a predetermined location.

Another embodiment includes a system for loading a biological sealant into a sealant dispensing device including: a headpiece having proximal and distal ends including: a first tubular barrel and second tubular barrel, each barrel disposed between the proximal and distal ends of the headpiece for transporting sealant materials, each barrel including an exit port at the distal end of the headpiece; a plunger extending into each barrel at the proximal end; a pressurized gas conduit disposed between the proximal and distal ends of the headpiece for transporting pressurized fluid, the gas conduit having a gas exit port at the distal end of the headpiece; and an attachment mechanism located at the distal end of the headpiece; a loader cartridge having a proximal end and a distal end including: an opening at the proximal end; a mating structure at the proximal end of the canister structure configured to be releasably engaged with the attachment mechanism of the head piece; and a plurality of open chambers within the loader cartridge, each open chamber being sized to house a vial in such a fashion that each vial is in fluid communication with one barrel exit port when the loader cartridge is connected to the headpiece.

The invention also includes an embodiment providing a method of loading biological sealant components including the steps of: providing a biological sealant dispensing apparatus including: a headpiece having proximal and distal ends including: a first tubular barrel and second tubular barrel, each barrel disposed between the proximal and distal ends of the headpiece for transporting sealant materials, each barrel including an exit port at the distal end of the headpiece; a plunger extending into each barrel at the proximal end; a pressurized gas conduit disposed between the proximal and distal ends of the headpiece for transporting pressurized fluid, the gas conduit having a gas exit port at the distal end of the headpiece; and an attachment mechanism located at the distal end of the headpiece; a loader cartridge having a proximal end and a distal end, including: an opening at the proximal end; a mating structure at the proximal end of the canister structure configured to be releasably engaged with the attachment mechanism of the head piece; and a plurality of open chambers within the loader cartridge, each open chamber being sized to house a vial in such a fashion that each vial is in fluid communication with one barrel exit port when the loader cartridge is connected to the headpiece; at least partially filling at least two vials with a biological material; attaching the loader cartridge to the headpiece; placing the at least two vials within the loading cartridge, such that one vial is placed within one open chamber, such that each exit port is in fluid connection with one of the vials; and drawing the biological material of each vial into the barrel with which it is in fluid connection by withdrawing the plurality of plungers in the proximal direction of the device.

### BRIEF DESCRIPTION OF THE FIGURES

FIG. 1 is a depiction of a prior art delivery device.

FIG. 2 is a depiction of one embodiment of the present invention having separate headpiece and applicator components.

FIG. 3 is a close up view of a headpiece useful in the present invention.

FIG. 4 is a view of an applicator useful in the present invention.

FIG. 5 is a perspective view of a dispensing conduit useful in the present invention.

FIG. 6 is a perspective view of a device useful in the present invention with optional cover removed from the applicator.

FIG. 7 is a perspective view of a headpiece with plungers completely removed from the barrels.

FIG. 8 is a sectional view of the components of one embodiment of the present invention, with each component being removed from the device.

FIG. 9A is a see-through view of a headpiece showing a gas line extending therethrough.

FIG. 9B is a cross-section side view of a headpiece useful in the present invention.

FIGS. 10A and 10B are end views of the headpiece with barrels completely withdrawn from the headpiece. FIG. 10A is in the “open” or “gas on” position while FIG. 10B is in the “closed” or “gas off” position.

FIG. 11 is a perspective view of a headpiece including a loading cartridge and detached vials.

FIG. 12A is a close up view of a headpiece including a see-through loading cartridge without vials. FIG. 12B shows a headpiece with vials loaded in a see through loading cartridge.

### DETAILED DESCRIPTION

With reference to the Figures, the present invention provides a gas-assisted delivery system for dispensing a fluid material, such as a sealant, with improved accuracy and ease of use. The system is useful for single-component sealants as well as multi-component sealants, including biological components that may either be applied directly or upon mixing with one or more additional components. The device is particularly useful for delivery of a multi-part biological sealant, such as fibrin. In such embodiments, the device contains and provides for mixing and delivery of a desired amount of fibrinogen and thrombin to the intended site. The device uses a pressurized flow of gas, such as air, carbon dioxide, or other gas, to aid in controlled delivery.

As used herein, the term “user” refers to the individual dispensing the biological material or sealant from the device. As used herein, the term “proximal” will refer to a location closest to the user dispensing the sealant from the device, e.g., the doctor or other medical professional. The term “distal” shall refer to a location closest to the delivery site, e.g., furthest away from the user dispensing the sealant from the device.

In recent years, delivery of biological components has been improved by using a gas-assisted delivery method, whereby the biological component or components is atomized by use of a pressurized gas line, and the atomized material is forced through a delivery port in the device. This forces the material in a controlled, precise manner. However, prior art devices suffer from a number of defects due in large part to the placement of the gas line. One such prior art device is seen in FIG. 1. FIG. 1 shows a prior art delivery device 10, having proximal end 10A and distal end 10B, the device 10 including two open chambers 12A/12B each having a plunger 14A/14B placed therein. Each plunger 14A/14B is slidably housed within an open chamber 12A/12B, where it may move along the axis of the open chamber 12A/12B in either a proximal or distal direction. As the user depresses the plungers 14A/14B, the material(s) stored in the chambers 12A/12B is through a delivery chamber 16, where they are forced out an open dispensing port 18 and allowed to be combined. At the same time, a gas line 22 delivers pressurized gas into the device at a gas entry port 20 in the delivery chamber 16. The gas is forced through delivery chamber 16, where it too exits at delivery port 18, where it atomizes the mixed composition.

While such devices are acceptable and deliver the material, they suffer from a number of drawbacks. For example, the gas line 22 is associated with the device 10 at gas entry port 20 that is close to the dispensing port 18, thus at least partially obstructing the user's view of the dispensing port 18 and the target area. Further, the gas line 22 enters the device at the delivery end, causing a lack of balance in using the device 10. In addition, as can be seen in FIG. 1, the gas line 22 interferes with the user's holding of the device 10, essentially limiting the placement of the user's hands on the device 10. Finally, as seen in FIG. 1, chambers 12A/12B are disposed in a parallel fashion. The gas line 22 enters the device 10 at an angle, which may disrupt the alignment of the device 10 and may throw off accurate mixing and delivery.

Thus, prior art devices such as those seen in FIG. 1 have the potential to not only disrupt the user's operation of the device 10 and thus the accuracy of delivery, but they have the potential to disrupt the mixing of the components and alignment of the device.

The present invention solves these and other problems. In one embodiment, a device of the present invention may be seen in FIGS. 2-10B. The device 100 as embodied in FIG. 2 is comprised of multiple components, including a headpiece 110 and an applicator 300, which are connectable via any secured connection. However, it is within the scope of this invention to have a single, unitary construction.

Headpiece 110 has a generally elongated shape, having a proximal end 110A and distal end 110B. Headpiece 110 includes a plurality of elongated open tubular or cylindrical chambers or barrels 120, which will be discussed in further detail below. There may be only one barrel 120, or there may be from about 2 to about 5 barrels in the device, depending upon the desired material(s) to be delivered. Each barrel 120 is disposed in a side-by-side manner, each barrel 120 being substantially parallel to each other, with their respective axes running from the proximal end 110A of the device to the distal end 110B of the device 100. Within each barrel 120 is disposed a plunger 130, which extends substantially the length of the barrel 120 and exits the barrel 120 at the proximal end 110A of the device. The plunger 130 is housed within a barrel 120 in such a fashion that it is movable in an axial fashion in the proximal 110A and distal 110B directions. In some embodiments, each plunger 130 may include a syringe piston 132, a portion of which is slidably positioned within the barrel 120. Each plunger 130 may also include a piston rod 134 attached to the syringe piston 132, and a syringe pusher 140 attached to the piston rod 134. Each pusher 140 is maintained outside the barrels 120, and allows the user to push or pull the associated plunger 130 as necessary, thus moving the plunger 130 axially either proximally or distally with respect to the barrel 120. Moving the plunger 130 proximally withdraws the plunger 130 out of the barrel 120, while moving the plunger 130 distally pushes the plunger 130 into the barrel 120. There may be multiple pushers 140 for each plunger 130, or each plunger 130 may be joined at a single pusher 140, which may be a separate piece or may be a unitary construction.

In some embodiments, the pusher(s) 140 may be curved or have ergonomic features to conform to a user's fingers, aiding in the comfort and ease of use. The pushers 140 may each be interconnected via a coupling unit, thus allowing for simultaneous pushing and pulling of each plunger 130 in the device, while in other embodiments the pushers 140 and plungers 130 may be made of a single construction. Each plunger 130 should form an air-tight seal within the barrel 120, thus pulling fluid within the barrel 120 when withdrawn axially out (proximally) and forcing fluid out of the barrel 120 when pushed axially in (distally). The air-tight seal may be achieved, for example, by using a deformable rubber or plastic piston 132, which is snugly fit within the interior of the barrel 120. The plungers 130 may be partially withdrawn from the barrels 120 by pulling the pusher(s) 140 axially in the proximal direction. As can be seen in FIG. 7, optionally, the plungers 130 may be completely withdrawn from the barrels 120 by pulling the pusher(s) 140 so that the plungers 130 are completely removed. In some embodiments, the plungers 130 may be fixed within the barrels 120, such that they cannot be completely removed therefrom. For example, there may be securement features, such as tabs or locks, in the back cover that restrict complete removal of the plungers 130 from the barrels 120. Such securement features may be, for example, an extending tab that engages with a portion of the plunger 130, such as its distal end, and prevents a user from inadvertently pulling the plungers 130 completely out of the barrel 120. Such restriction aids in preventing accidental opening of the barrel 120 and spilling its contents.

With reference to FIG. 3, at the distal 110B end of the headpiece 110 each barrel 120 is in fluid association with a barrel exit port 160A/160B. The barrel exit port 160 may be any desired shape or size, including cylindrical, and may include a gasket or other sealing mechanism. The barrel exit port 160 should be in an air-tight engagement with the barrel 120 with which it is associated. As such, any fluid that is drawn into the barrel 120 travels through the barrel exit port 160, and any fluid that exits the barrel 120 travels through the barrel exit port 160. It is conceivable that a barrel exit port 160 may be associated with more than one barrel 120, but in preferred embodiments, one barrel exit port 160 is in fluid association with its own barrel 120. The barrel exit port 160 preferably has a slimmer configuration than the barrel 120 with which it is associated. Barrel exit port 160 may include a rubber gasket around its periphery.

In embodiments in which the headpiece 110 and the applicator 300 are separate and distinct components, the distal end 110B of the headpiece contains an attachment mechanism 150. The attachment mechanism 150 may be any suitable device to attach two pieces together, including, for example, a clip-type mechanism, snap fit design, spring loaded design, or a force or friction-fit mechanism. The attachment mechanism 150 may be any size or shape desired, and should be sized accordingly to allow attachment to the applicator 300, as will be described in further detail below.

The headpiece 110 and its components may be made of any desired material, including, for example, plastic, glass, rubber, and combinations thereof. It is particularly desirable that the headpiece 110 and its components be made of a biologically inert material, thus allowing the device 100 to be suitable for the delivery of biological materials to an individual. The barrel 120 may be made of a transparent or translucent material, thus allowing the user to visually inspect the contents in the barrel 120. Further, the use of a transparent or translucent material allows the user to view the plunger 130 as it pushes or pulls axially through the barrel 120, either drawing fluid into the barrel 120 or forcing fluid out of the barrel 120. The barrel 120 may contain markings, such as graduated markings, so as to allow the user to visually inspect the level of material in the barrel 120. Each barrel 120 in the device 100 may be any desired size to allow the proper amount of fluid to be delivered. In some embodiments, each barrel 120 has the same diameter and axial length, while in other embodiments, at least one barrel 120 has a larger or smaller diameter and/or a larger or smaller axial length. In embodiments where a high fibrin ratio is to be dispensed, for example, the composition includes a greater amount of fibrinogen than thrombin, and thus a first barrel 120 including fibrinogen may have a greater size (volume) than the second barrel 120 including thrombin. Each barrel's 120 plunger 130 may be depressed at the same time, speed, pressure, and rate, but due to the difference in volume of the barrels 120, varying amounts of materials can be dispensed simultaneously.

The headpiece 110 further includes a gas entry port 180 at its proximal location, and a gas exit port 170 at its distal location. The gas entry port 180 and gas exit port 170 are in fluid communication via a generally tubular gas line 200. The gas line 200 runs from the proximal end 110A to the distal end 110B of the headpiece 110, and is substantially parallel to the barrels 120. The position of the gas line 200 in the headpiece can best be seen in FIGS. 8-9B. In some embodiments, the gas line 200 is disposed at a position that lies in between at least two barrels 120, although it may be disposed at any location in the device 100. The gas line 200 may have the same diameter as the barrels 120, or it may have a circumference that is smaller or larger. It may be desirable that the gas line 200 have a smaller diameter than the barrels 120.

At the proximal end 110A of the headpiece 110, there is disposed a gas entry port 180. The gas entry port 180 may be any size or shape and is intended to be securely attached to a gas line 200 via any secure means, such as by using connector 240. A gas feed tube (not pictured) may run from an external gas supply (not shown) to the device 100, so as to transport gas, such as air, carbon dioxide, or other gas, to the device 100. The gas supply may be any desired container or known device for supplying air or other inert or stable gas, including a tank or blower. The gas supply may be a portable device, which may be moved by the user or held by the user during use of the device. The gas supply may be capable of being turned on and off by a user with ease, such as by a hand or foot activation.

The headpiece 110 may also include an actuator 190 to control the flow of fluid, such as gas, into the device 100. One exemplary actuator 190 can be seen in FIGS. 7-10. For example, the device 100 may include a rotational actuator 190, whereby rotation thereof allows or stops the flow of gas along the gas line 200. The use of this actuator system allows operation of the flow of gas by the user's finger. In some embodiments, gas flow actuation may be achieved by a movement in a direction that is substantially different from the dispensing direction, e.g., a slight sideways motion. As can best be demonstrated in FIGS. 10A and 10B, the actuator 190 may be rotated in a sideways motion (or alternatively in a clockwise/counterclockwise motion) to control the flow of gas. The actuator 190 includes a first arm 210 and a second arm 220, which extend into the housing of the headpiece 110. The rotation of the actuator 190 may be achieved through the use of a post 230, which is fit into the actuator 190 and allows for rotation of the actuator 190 along its axis. By moving the actuator 190 in either direction, the second arm 220 may engage the gas line 200, pinching the gas line 200 and stopping the flow of gas therethrough. In FIG. 10A, the actuator 190 is in the “open” or “gas on” position, where the arm 220 is not engaged with the gas line 200 and does not stop the flow of gas. In FIG. 10B, the actuator 190 is in the “closed” or “gas off” position, and it can be seen that second arm 220 pinches the gas line 200, thus blocking the flow of gas therethrough. Arm 210 is included to hold the actuator 190 in either position and may also optionally include detents to provide tactile or audible feedback to the user confirming actuator position. The actuator 190 may be biased in any direction, for example, it may be biased into the closed position. A cover plate 250 may be used to keep the components in place.

Although the exact placement of the gas entry port 180 and actuator 190 may be modified, it is particularly desirable that the gas entry port 180 and the actuator 190 both be located at or near the proximal end 110A of the headpiece 110. Location at the proximal end 110A allows for greater control and less obstruction to the user during use. In addition, by placing the gas entry port 180 and actuator 190 at the proximal end 110A of the headpiece 110, the gas line 200 need not be disrupted, disconnected or otherwise altered if the applicator 300 is removed, changed or modified during use. By maintaining the gas entry and control on the headpiece 110, less manipulation of components is needed before and during use. Further, by placing the gas entry port 180 and actuator 190 at the proximal end 110A, greater user control of flow and pressure can be achieved.

When the actuator 190 is in an open position, e.g., allowing the flow of pressurized gas into the device 100, the gas flows along the gas line 200 to the gas exit port 170, where it is exerted from the headpiece 110 under pressure. During use, the desired pressure of fluid, such as air or other inert or stable gas, into the device is about 20-25 psi. As the gas travels from the entry port 180, through the line 200 and into the applicator 300, it necessarily loses pressure but maintains a sufficient velocity to atomize the liquid materials as the gas exits the device 100 through applicator 300 via applicator tip 410. Suitable velocity allows the gas to sufficiently atomize the biological components during use, providing controlled delivery. As the various components exit the device 100, the rate of speed of fluids is about 50-150 msec, and more particularly about 100 msec. Of course, the viscosity of the materials to be dispensed may alter the final velocity. Pressure and velocity may be controlled by manual manipulation by the user, e.g., opening or closing a valve, adjusting a pressure regulator, or may be controlled electronically by using an electronic valve system. Pressure monitors may be used to communicate the pressure level of air or other inert or stable gas to the user or a computer system.

The headpiece 110 is used in conjunction with an applicator 300. With reference to FIG. 4, a separate applicator 300 is shown, but as stated above, the headpiece 110 and applicator 300 may be a single construction if desired. The applicator 300 may be made from the same materials as the body of the headpiece 110, or it may be made of different materials. Similarly, however, the applicator 300 should be made from biologically stable and inert materials so as to avoid contamination of the biological sealant to be delivered. The applicator 300 has a proximal end and distal end, as defined above. The applicator 300 may optionally include a substantially hollow cover 310, which serves as a casing to protect the components housed within and also allows for attachment of the applicator 300 to the headpiece 110. The optional cover 310 may be removable if desired.

In embodiments in which the applicator 300 and headpiece 110 are separate pieces, the proximal end of the applicator 300 includes a mating structure 320, which is sized and shaped to allow for a secure fit with the attachment mechanism 150 of the headpiece 110. As explained above, any desired attachment mechanism may be used, so long as the fit between the mating structure 320 and the attachment mechanism 150 is secure, and desirably fluid-tight. A gasket or other securement means such as a radial seal may be included in the mating structure 320 and/or the attachment mechanism 150 so as to provide a more fluid-tight seal. The mating structure 320 and attachment mechanism 150 are aligned such that there is a fluid connection between the passageways in the headpiece 110 and in the applicator 300, as will be explained below.

Within the body of the applicator 300, there may be a series of open tubular structures or fluid passageways. Any number of fluid passageways may be used, and desirably there is one fluid passageway for each exit port in the headpiece 110. For example, there may be three fluid passageways 330, 340, 350, which are each in fluid communication with at least one exit port in the headpiece 110. In some embodiments, each fluid passageway (e.g., 330, 340, 350) may extend from the proximal end of the applicator 300 to the distal end of the applicator 300. Each fluid passageway 330/340/350 is sized and shaped to fluidly join with one of the exit ports 160/170 of the headpiece 110, and should be sufficiently sized to allow the various fluids, such as gas, sealant, or biological materials, to travel once they are released from the headpiece 110. There is desirably one tubular structure (passageway) associated with and in fluid communication with each barrel exit port 160, and another tubular structure (passageway) associated with and in fluid communication with gas exit port 170. When the applicator 300 is connected to the headpiece 110, as seen in FIG. 6, fluid passageway 330 mates with barrel exit port 160A, fluid passageway 340 mates with barrel exit port 160B, and fluid passageway 350 mates with gas exit port 170. The three fluid passageways 330/340/350 may converge to a convergence point 360, which leads to a dispensing structure, such as dispensing conduit 400. Dispensing structure need not be a tubular conduit 400, and may simply include an opening or port at the end of the convergence point 360, or other dispensing structure, such as a brush or applicator. The dispensing structure may, for example, be an open nozzle, but may alternatively include an application means, such as a spatula, rolling ball, brush, and/or swab. Any dispensing and application system may be used, including those described in U.S. Pat. No. 6,425,704, the entire content of which is incorporated by reference herein. The Figures shown herein depict a dispensing conduit 400, but it should be understood that dispensing structure is not limited to a tubular conduit, and if a conduit 400 is used, it may have any length and diameter desired.

The distal end of the applicator 300 may include a dispensing conduit 400, which may be a generally cylindrical component and may extend out from the applicator 300, allowing for controlled dispensing and delivery of the fluid components to the desired site. One embodiment of a dispensing conduit 400 can best be seen in FIG. 5, and it should be understood that dispensing conduit 400 may have any length or diameter, and may be tapered if desired. In some embodiments, the dispensing conduit 400 may be a generally tubular body having a plurality of open passageways extending from the convergence point 360 to a dispensing tip 410. The dispensing conduit 400 may include a plurality of open passageways, such as three open passageways 420/430/440, each of which extends along the length of the dispensing conduit 400. The proximal end of the dispensing conduit 400 is in fluid communication with the end of each of the fluid passageways 330/340/350 at convergence point 360. In desired embodiments, there is one open passageway (e.g., 420) for each fluid passageway (e.g. 330), with a fluid association therewith, allowing the flow of fluid from fluid passageway (e.g. 330) to the open passageway with which it is associated (e.g., 420). The fluid connection therebetween should be fluid tight and secure enough to withstand pressure generated by the flow of fluids. In some embodiments, the fluid passageways 330/340/350 and open passageways 420/430/440 are made of a unitary construction, while in other embodiments, the dispensing conduit 400 may be removable from the applicator 300.

In embodiments including a dispensing conduit 400, the dispensing conduit 400 may have a plurality of openings 450/460/470 at its dispensing tip 410, each opening 450/460/470 being the end of one of the open passageways 420/430/440, and each opening is sized and configured to allow for the desired amount of material to be dispensed. The openings 450/460/470 in dispensing tip 410 may be any shape or size desired, and may simply be open circular cross section. In some embodiments, the openings 450/460/470 in dispensing tip 410 may have an oval or square cross section to provide longitudinal delivery of components. In some embodiments, the dispensing tip 410 may have an applicator feature, such as a brush or nozzle, and it may be flared or tapered.

In this embodiment, fluids are capable of being forced from the fluid passageways 330/340/350 through the open passageways 420/430/440 in the dispensing conduit 400, and exiting at one of the openings 450/460/470 in the dispensing tip 410. The dispensing conduit 400 may be made from any desired biologically stable and inert materials. In some embodiments, it may be preferable that the dispensing conduit 400 be substantially rigid, while in other embodiments, the dispensing conduit 400 may be flexible enough to allow a user to flex the conduit 400 with his or her hand. In use, the fluids, including biological materials and gas, exit their respective openings 450/460/470 in the dispensing tip 410 simultaneously, at which time mixing and atomization of the mixed composition occurs.

During use, fluids are expressed through the barrels 120 through use of plungers 130 and simultaneously gas is flowed through gas line 200. The fluids (including gas) each travel through respective exit ports 160/170, where they enter one of the fluid passageways 330/340/350. The fluids (including gas) travel through their respective fluid passageways 330/340/350, where they enter one of the open passageways 420/430/440 and travel through open passageways 420/430/440 along the length of the dispensing conduit 400. Finally, the fluids (including gas) exit the device 100 at one of the openings 450/460/470, where the materials are mixed and atomized, and dispensed at the desired location.

In some embodiments, the barrels 120 may include an interior drive track or other alignment means, so as to ensure alignment with the plunger 130 associated therewith. For example, the inner surface of a barrel 120 may include an indented portion along its length, while the plunger 130 associated with that barrel 120 may include a raised portion sized to fit within the indented portion.

The device 100 may be used to deliver materials, such as biological materials, including sealant materials, to an intended site. The site may be any intended area where delivery of such materials is desired, including, for example, open wounds or other biological tissue. During use, sealant materials are initially contained within the plurality of barrels 120, and the plungers 130 associated with the barrels 120 are fully extended out the proximal end 110A of the headpiece 110. This configuration is termed the “ready” or “loaded” configuration, which means that the materials contained within the barrels 120 are ready to be dispensed by the user. It is possible that the barrel exit ports 160 may be covered or sealed with a cap or other covering means until ready for use, although typically the materials to be dispensed are dispensed soon after being loaded into the headpiece 110.

In one embodiment, there are two barrels 120 in the device 100, a first barrel 120A and a second barrel 120B. The first barrel 120A contains a first biological material, and the second barrel 120B contains a second biological material. The first biological material and second biological material may be the same or they may be different. For example, the first barrel 120A may contain fibrinogen, and the second barrel 120B may contain thrombin. When mixed, these biological materials form fibrin, which is a desired biological sealant. Any materials that are intended to be mixed with each other may be used, if desired. For example, the device may be useful in delivering other adhesives that may not include biological materials, such as acrylates, if desired. Any materials to be combined and/or atomized may be used with the present device 100. Each barrel 120 contains a desired amount of biological material, and the barrel 120 need not be completely filled with biological material. In some embodiments, the amount of the biological material in each barrel 120 is substantially equal.

For embodiments in which fibrin is delivered, a first barrel 120 includes first fluid composition including a desired amount of fibrinogen and a second barrel 120 includes second fluid composition including a desired amount of thrombin. The fluid composition including fibrinogen (housed in and released from the one barrel 120) may be used in any amount desired, and most desirably between about 0.1 cc to about 5.0 cc, and more desirably from about 1.0 cc to about 5.0 cc. The fluid composition including thrombin (housed in and released from another barrel 120) may have a volume of about 1 to about 1/40 the amount of the fluid composition including fibrinogen, and more particularly from about ⅓ to about 1/10 the amount of the fluid composition including fibrinogen. That is, the amount of thrombin to be delivered may be less than the amount of the fibrinogen to be delivered. In such embodiments, the barrel 120, which houses the fibrinogen, may have a greater volume than the other barrel 120, which houses the thrombin. Each plunger 130 within the barrels may be depressed at the same time, rate, speed, and pressure, but due to differing volumes, different amounts of each fluid may be dispensed at the same time. In some embodiments, one barrel 120 may have a volume that is about 1 to about 40 times as great, and may have a volume that is about 3 to about 10 times as great as another barrel 120.

In embodiments where there is a separate headpiece 110 and applicator 300, the headpiece 110 and applicator 300 are secured to each other before delivery. In this embodiment, the mating structure 320 and the attachment mechanism 150 are connected, providing a secure and tight connection between the headpiece 110 and applicator 300, and aligning the fluid passageways 330/340/350 of the applicator 300 with one exit port each (either barrel exit port 160 or gas exit port 170). When connected, there is a fluid association between the openings of the barrels 120 and the fluid passageways 330/340 in the applicator 300. The fluid passageways 330/340 extend along the open cover 310, and enter the open passageways 420/430 of the dispensing conduit 400 at convergence point 360. Thus, there is a secure, fluid connection from the open interior of each barrel 120, through an exit port 160, through a fluid passageway 330/340, and out a dispensing structure. The fluid connection may extend through an open passageway 420/430 of a dispensing conduit 400 and out an opening 450/460 in the dispensing tip 410. There is also a secure, fluid association between the gas entry port 180 through gas line 200, through gas exit port 170, through fluid passageway 350, which may extend through open passageway 440 in a dispensing conduit 400, and out opening 470 in the dispensing tip 410. In embodiments where a dispensing structure is not an extended dispensing conduit 400, the fluid and/or gas may be dispensed through any desired dispensing structure.

The present invention further includes a method of using the device 100 to deliver a fluid composition to an intended site of delivery. The discussion herein will entail delivery of a two-part composition, including a first fluid composition and a second fluid composition, but it will be understood that the method described herein may be used to deliver a single-part composition or a composition that entails more than two separate components. In preferred embodiments, the first fluid is thrombin and the second fluid is fibrinogen, which are mixed together to form fibrin. Again, other, non-biological materials may be delivered with the present invention, including multi-part sealants such as acrylates or the like.

The device 100 includes the components described above, including a headpiece 110 and applicator 300 with dispensing conduit 400. The headpiece 110 and applicator 300 may be separate pieces that are attachable to each other, or they may be a single unitary piece. As explained above, the headpiece 110 includes at least one barrel 120, and desirably two barrels 120, where each barrel 120 is sized and fit to house a fluid composition to be delivered. In some embodiments, a first barrel 120 may have a greater volume than a second barrel 120, to allow for differing levels of fluid to be ejected therefrom simultaneously. The device 110 is placed in the “ready” position, as explained above. That is, each barrel 120 includes a desirable amount of fluid to be delivered, the plungers 130 are each pulled axially in the proximal direction 110A, and the device is ready to dispense the composition. The applicator 300 is secured to the headpiece 110, thereby aligning and providing a secure fluid connection between the barrels 120 and the fluid passageways 330/340, and a secure fluid connection between the gas line 200 and fluid passageway 350.

While in the “ready” position, the user secures a gas feed line (not shown) to the gas entry port 180. A biologically stable and inert gas, such as air, carbon dioxide or other gas, is allowed to flow under pressure through the gas feed line towards the gas entry port 180. The actuator 190 is maintained in the closed position, thus pinching the gas line 200 with the second arm 220 of the actuator 190. Either prior to commencing delivery of the material to be delivered, or simultaneous with delivery, the user rotates the actuator 190, thus releasing the clamp on the gas line 200, and allowing the gas to flow along the gas line 200 and exiting the gas exit port 170. During use, the gas is initially pressured to a level of about 20-25 psi and has a velocity of about 50-150 msec. As the gas exits the gas exit port 170 into the applicator 300, the velocity is about 50-150 msec.

The user aligns the device 100 at the intended site of delivery, e.g., aligning the dispensing tip 410 of the dispensing conduit 400 at the intended site of delivery. The user then depresses the plungers 130 at a sufficient pressure to force the fluid materials from each barrel 120 out of their respective exit ports 160, through their respective fluid passageways 330/340, along their respective open passageways 420/430 in the dispensing conduit 400, and out their respective openings 450/460 in dispensing tip 410. Upon dispensing, the fluid materials are mixed and delivered.

As the plungers 130 are being depressed, pressurized air or carbon dioxide (or other stable or inert gas) is flowing through the gas line 200, out the gas exit port 170, along the gas passageway 350 of the applicator 300, and along passageway 440 in the dispensing conduit 400, where it is released through opening 470 in dispensing tip 410. In some embodiments, the gas from the gas line 200 is of a sufficient velocity to atomize the fluid materials upon exit from the device 100, forming an atomized mixture. If each of the plungers 130 is depressed at the same time, the common depressing of the plungers 130 allows the contents of the separate components to be expressed, dispensed or exhausted separately but simultaneously. With the concurrent flow of pressurized gas out of the dispensing tip 410, the mixed material may be atomized in droplet form.

The user may continue to depress the plungers 130 at the intended rate and pressure to provide for delivery of the atomized mixture to the site of delivery for the length of time needed to deliver a sufficient amount of materials. During delivery, the user may move the device 100 as desired to ensure delivery of the atomized mixture to an intended region. Once the sufficient amount of atomized mixture is delivered to the intended site, the user may cease pressing on the plungers 130, shut the actuator 190, and/or stop the flow of gas from the gas supply, thereby stopping the flow of atomized mixture through the dispensing tip 410.

Further, at any time during use, the user may temporarily cease pressing on the plungers 130, shut the actuator 190 into the “closed” position, and/or stop the flow of gas from the gas supply to stop the flow of mixed composition, so that the user may move the device 100 to a different intended delivery site or pause for any desired reason. For example, the user may cease pressing on the plungers 130, move the actuator 190 to the closed position, and/or stop the flow of gas from the gas supply in the event that the device needs to be re-loaded with fluid.

Once delivery of the fluid components is completed, the user may optionally disconnect the gas feed line (not shown), and either sanitize the various components of the device 100 or simply discard the components that were in contact with biological materials.

As set forth in FIGS. 11-12, the present invention also provides an apparatus and method for loading fluid materials into the device 100, and more particularly for loading biological materials into the barrels 120 of the device. Fluid materials, particularly biological materials, are commonly stored in vials, the interior of which is maintained in a sterile fashion. The outside of the vials, however, may not be sterile, due to storage and handling. In typical loading methods, the biological materials are emptied from the vial into a loading cup, and drawn into the barrel with which it is to be loaded. This may cause a lack of sterility, and also requires the users loading the device to be cautious and careful in loading. The present loading apparatus and method provides a means for ensuring a higher degree of sterility, thus allowing for the biological materials to be loaded into the device 100 while avoiding contamination of the materials. The loading system and apparatus of the present invention is particularly useful for embodiments in which there is a separate headpiece 110 and applicator 300. In this embodiment, the applicator 300 is kept separate from the headpiece 110 until loading is complete.

FIGS. 11 and 12A-B depict one embodiment of a loading system for the device 100. The loading system may be useful in loading materials, and particularly biological materials into the barrels 120 of the device, thus putting the device in the “ready” state. Further, a loading system may be in the process of delivery of the biological material, for example, in the event that the barrels 120 have expelled biological materials and the user wishes to re-fill the barrels 120 during use.

In this embodiment, the device 100 as explained above is provided with a loading cartridge 500. The loading cartridge 500 is a generally hollow device which has a plurality of open loading chambers 510, sized and shaped to house a plurality of vials 550A/550B. The loading cartridge 500 includes a proximal end 500A and distal end 500B. Each loading chamber 510 is generally tubular in structure and is sized to house a vial 550, which is desirably smaller than the loading chamber 510 into which it is to be placed. The loading chambers 510 may have the same or may have differing volumes, diameters, or axial lengths. In preferable embodiments, there may be the same number of loading chambers 510 in the loading cartridge 500 as there are barrels 120 in the headpiece 110. That is, if the headpiece 110 includes two barrels 120, the loading cartridge 500 may include two loading chambers 510, each intended to house one vial 550 of biological material. Further, the loading chambers 510 are aligned in substantially the same fashion in the loading cartridge 500 as the barrels 120 are in the headpiece 110. That is, if the headpiece 110 includes two barrels 120 in a side-by-side configuration, the loading cartridge 500 will include two loading chambers 510 in a side-by-side configuration.

The proximal end 500A includes a coupling adaptor 540, which is sized and shaped to mate with and form a secure connection with the attachment mechanism 150 of the headpiece 110. Thus, if the attachment mechanism 150 is a snap-fit design, the coupling adaptor 540 will have the corresponding snap-fit design, ensuring a locked connection between the two components: headpiece 110 and loading cartridge 500. The coupling of the headpiece 110 and loading cartridge 500 desirably provides a secure, fluid-tight fit. A gasket or other securement means such as a radial seal may be included in the coupling adaptor 540 and/or the attachment mechanism 150 so as to provide a substantially fluid-tight seal.

The distal end 500B of the loading cartridge 500 may include a cover 520, which covers a distal opening of the loading cartridge 500. Any type of cover 520 may be used, and in some embodiments, the cover 520 is secured to the loading cartridge 500 via a hinge 530. In this fashion, the cover 520 may be secured or opened with ease and without fear of misplacing cover 520. The cover 520 may have a secure locking mechanism so as to restrict opening once it has been secured in place. Further, cover 520 may include a gasket or other sealing means to secure an air tight seal when closed.

In some embodiments, the vials 550 may be separate and removable from the loading cartridge 500. In other words, the user may insert or remove vials 550 into or from the loading chambers 510 of the loading cartridge 500. The cover 520 may simply be detached from the distal end of the cartridge 500, and the vials 550 inserted into the cartridge 500 by a user. To provide a secure fit, the cover 520 may then be resecured to the cartridge 500 after vials 550 are inserted. This embodiment may allow for easier filling, replacement, or disposal of the vials 550. The vials 550 and loader cartridge 500 may include a locking mechanism for securing vials 550 in place, such as a snap fit, spring loading, or friction fit mechanism in the loading chambers 510. In some embodiments, it may be desired that the cover 520 be securely fastened after loading the vials in place, such that the cover 520 cannot be easily or unintentionally removed once secured. The loading cartridge 500 may be at least partially transparent or translucent so as to allow a user to view the interior thereof.

Each vial 550 includes a pre-determined amount of biological material, and includes an open proximal end 560, which may be covered with a cap or other sealing device. Each vial 550 is sized to be placed into one of the loading chambers 510, such that the proximal opening 560 of the vial 550 is placed at the proximal end of the loading chamber 510. After the vial 550 is placed into the loading cartridge 500, the cover 520 may be closed. Each loading chamber 510 may include a track or other means to align the vial 550 with a piercing element or an exit port 160. In this fashion, each opening 560 of the vials 550 is aligned with one exit port 160 of the headpiece 110 when the components are secured to each other. Thus, when the headpiece 110 is secured to the loading cartridge 500, one exit port 160 is in fluid communication with the proximal opening 560 in one vial 550. The exit port 160 is thus in fluid communication with the interior of the vial 550, and has access to the biological materials housed therein, without fear or risk of having the exit port 160 contact the exterior of the vial 550, which may not be a sterile environment. The interior of the loading chambers 510 may be adapted to include one or more piercing elements, which allows for a cover or septum covering the vial to be pierced and allow the contents therein to be loaded into the barrel(s).

The invention provides a method of loading the headpiece 110 to place the device in the “ready” state. When in the “ready” state, the barrels 120 of the headpiece 110 contain a sufficient amount of material, such as biological material, to deliver the intended final product. The headpiece 110 as explained above is provided, where each barrel 120 is substantially free of biological material, or if additional biological materials are needed in the barrels 120. Each plunger 130 is depressed into the barrel 120 with which it is associated. A loading cartridge 500 and a plurality of vials 550 are provided, each vial 550 having a desired amount of biological material therein. In some embodiments, the biological material in each vial 550 may be the same or may be different. Desirably, each vial 550 includes a separate biological material, which provides a desired mixed composition when the biological materials are mixed. For example, a first vial 550A may include fibrinogen and a second vial 550B may include thrombin.

The attachment mechanism 150 of the headpiece 110 and the coupling adaptor 540 of the loading cartridge 500 are secured to each other, forming a secure fit. The vials 550 are each placed into one of the loading chambers 510 of the loading cartridge 500, such that the proximal opening 560 in each vial 550 is in fluid communication with the exit port 160 with which it is associated. Once connected and secured, the user withdraws the plungers 130 in the proximal direction (e.g., 110A), thereby drawing the biological material from the vial 550 into the barrel 120 with which it is in fluid communication, via exit port 160. When a desired amount of biological material is drawn into the barrel 120, the user ceases withdrawing the plunger 130.

The vials 550 and/or loading cartridge 500 may then be detached from the headpiece 110, if desired. The distal end 110B of each barrel 120 may optionally be sealed by the user with a cap or other cover and stored until ready to be used. When the device 100 is ready to be used, the user may remove the cap or cover from the distal end 110B of each barrel 120, and then connect the applicator 300 to the headpiece 110, as explained above. Of course, the applicator 300 may be connected to the headpiece 110 immediately after removal of the loading cartridge 500. With the desired amount and type of fluids in the device 100, the device 100 is now in the “ready” configuration, since there is a sufficient amount of material within the barrels) 120 of the device 100. If not already connected, the user may connect the applicator 300 to the headpiece 110, and secure a gas feed line to the gas entry port 180, as explained above, and deliver the biological materials as explained above.

The easy removal and securement of both the applicator 300 and the loading cartridge 500 to the attachment mechanism 150 of the headpiece allows a user to quickly and efficiently remove the applicator 300 during use, replacing it with a loading cartridge 500 so as to fill the barrels 120, and then re-place the applicator 300 to continue dispensing material. In addition, the securement described above provides a method of loading and dispensing of material while maintaining a required level of sterility to the materials to be delivered. This is particularly important when biological materials are to be delivered. Since the gas connection is located on the proximal end of the headpiece 110, no manipulation or movement of the gas feed line is required when components are changed, secured or detached.

The device 100 may be used with different types of dispensing conduits 400 and/or tips 410 other than that described above. Since the headpiece 110 and applicator 300 are separable pieces, different applicators may be secured to the headpiece 110 as necessary.

The device 100 may be provided in a kit, which includes the device 100 as explained above and one or more loading cartridges 500 and/or vials 550. The vials 550 may be pre-filled with fluid or they may be free of deliverable fluid. The kit may be provided with the headpiece 110 and applicator 300 as separate pieces, or they may be secured to each other. Further, the kit may be provided with different dispensing conduits 400 or with different styles of applicators 300, each of which is securable to the headpiece 110. The kit may further include a gas supply and/or gas line 200. The kit may optionally include a set of instructions for connecting the various components and using the device 100.

Other variations may include, for example, the use of an automated system for depressing the plungers 130, such as a spring loaded system or electronically-driven system, rather than the manual depression by a user. The plungers 130 may alternatively be controlled by a rotational mechanism, such as a screw-type system, in which rotation in a first direction moves the plunger 130 axially in the proximal direction and rotation in a second direction moves the plunger 130 axially in the distal direction.

What is claimed is:

1. A device for dispensing a biological sealant comprising:
a. a headpiece having proximal and distal ends comprising:
i. a first tubular barrel and second tubular barrel, each barrel disposed between the proximal and distal ends of the headpiece, each barrel including an exit port at said distal end of the headpiece;
ii. a plunger extending into the proximal end of each barrel;
iii. a pressurized gas conduit disposed between the proximal and distal ends of the headpiece, said gas conduit having a gas entry port at the proximal end of the headpiece and a gas exit port at the distal end of the headpiece; and
iv. an attachment mechanism located at the distal end of the headpiece; and
b. an applicator having proximal and distal ends, comprising;
i. a mating structure at the proximal end of the applicator, the mating structure configured to be releasably engaged with the attachment mechanism on the headpiece;
ii. a dispensing structure disposed at the distal end of the applicator;
iii. a first and second fluid passageway, each fluid passageway being in fluid communication with one of said exit ports; and
iv. a first gas passageway in fluid communication with said gas exit port; and
c. an actuator located on the headpiece for controlling pressurized fluid flow through the pressurized gas conduit.

2. The device of claim 1, wherein said biological sealant is fibrin.

3. The device of claim 2, wherein at least one tubular barrel comprises fibrinogen and at least one tubular barrel comprises thrombin.

4. The device of claim 1, wherein each plunger comprises: a syringe piston, a portion of which is slidably positioned within a barrel, a piston rod attached thereto, and a syringe pusher attached to the piston rod.

5. The device of claim 1, wherein each barrel comprises a drive track aligning a plunger disposed within said barrel.

6. The device of claim 5, wherein said plungers are connected to each other via a coupling unit at a proximal location of the device.

7. The device of claim 1, wherein said plungers and said pressurized gas conduit may be used simultaneously to dispense multiple fluids concurrently.

8. The device of claim 1, further comprising a gas supply source for providing gas to said pressurized gas conduit.

9. The device of claim 1, wherein said actuator comprises at least one rotatable arm for clamping said pressurized gas conduit.

10. The device of claim 1, wherein said first and second fluid passageways extend from said proximal end to said distal end of said applicator.

11. A method of delivering a biological material to a delivery site, comprising the steps of:
a. Providing a device for dispensing a biological material, the device comprising:
i. a headpiece having proximal and distal ends comprising:
1. a first tubular barrel and second tubular barrel, each barrel disposed between the proximal and distal ends of the headpiece for transporting sealant materials, each barrel including an exit port at said distal end of the headpiece;
2. a plunger extending into each barrel at said proximal end;
3. a pressurized gas conduit disposed between the proximal and distal ends of the headpiece for transporting pressurized fluid, said gas conduit having a gas exit port at the distal end of the headpiece; and
4. an attachment mechanism located at the distal end of the headpiece;
ii. an applicator having proximal and distal ends, comprising;
1. a mating structure at the proximal end of the applicator, the mating structure configured to be releasably engaged with the attachment mechanism on the headpiece;
2. a dispensing structure disposed at the distal end of the applicator;
3. a first and second fluid passageway, each fluid passageway being in fluid communication with one of said exit ports and terminating at said dispensing structure;
4. a first gas passageway in fluid communication with said gas exit port; and
iii. an actuator located on the headpiece for controlling pressurized fluid flow through the pressurized gas conduit;
b. at least partially filling said first barrel with a first biological material and at least partially filling said second barrel with a second biological material;
c. moving each of said plungers in a distal direction so as to force said first biological material from said first barrel through said first fluid passageway and said second biological material from second barrel through said second fluid passageway;
d. moving said actuator so as to permit the flow of pressurized gas to travel through said gas conduit through said first gas passageway;
e. atomizing said biological material to form an atomized composition; and
f. dispensing said atomized composition at a predetermined location.

12. The method of claim 11, wherein said first biological material and said second biological material are different materials.

13. The method of claim 12, wherein a first biological material is thrombin and a second biological material is fibrinogen.

14. The method of claim 11, wherein said gas is air.

15. The method of claim 11, wherein said first and second fluid passageways extend from said proximal end to said distal end of said applicator

16. A system for loading a biological sealant into a sealant dispensing device comprising:
a. a headpiece having proximal and distal ends comprising:
i. a first tubular barrel and second tubular barrel, each barrel disposed between the proximal and distal ends of the headpiece for transporting sealant materials, each barrel including an exit port at said distal end of the headpiece;
ii. a plunger extending into each barrel at said proximal end;
iii. a pressurized gas conduit disposed between the proximal and distal ends of the headpiece for transporting pressurized fluid, said gas conduit having a gas exit port at the distal end of the headpiece; and
iv. an attachment mechanism located at the distal end of the headpiece;
b. a loader cartridge having a proximal end and a distal end comprising:
i. an opening at the proximal end;
ii. a mating structure at the proximal end of said canister structure configured to be releasably engaged with the attachment mechanism of the head piece; and
iii. a plurality of open chambers within said loader cartridge, each open chamber being sized to house a vial in such a fashion that each vial is in fluid communication with one barrel exit port when the loader cartridge is connected to the headpiece.

17. The system of claim 16, comprising a releasable cover at the distal end of the loader cartridge, wherein at least one open chamber may be securely closed when said cover is secured to the loader cartridge.

18. The system of claim 16, wherein the loader cartridge contains at least one piercing element.

19. The system of claim 18, wherein the open chamber comprises a track to align a vial with at least one of a piercing element and an exit port.

20. The system of claim 16, wherein said loader cartridge is at least partially transparent.

21. A method of loading biological sealant components comprising the steps of:
a. Providing a biological sealant dispensing apparatus comprising:
i. a headpiece having proximal and distal ends comprising:
1. a first tubular barrel and second tubular barrel, each barrel disposed between the proximal and distal ends of the headpiece for transporting sealant materials, each barrel including an exit port at said distal end of the headpiece;
2. a plunger extending into each barrel at said proximal end;
3. a pressurized gas conduit disposed between the proximal and distal ends of the headpiece for transporting pressurized fluid, said gas conduit having a gas exit port at the distal end of the headpiece; and
4. an attachment mechanism located at the distal end of the headpiece;
ii. a loader cartridge having a proximal end and a distal end, comprising:
1. an opening at the proximal end;
2. a mating structure at the proximal end of said canister structure configured to be releasably engaged with the attachment mechanism of the head piece; and
3. a plurality of open chambers within said loader cartridge, each open chamber being sized to house a vial in such a fashion that each vial is in fluid communication with one barrel exit port when the loader cartridge is connected to the headpiece;
b. at least partially filling at least two vials with a biological material;
c. attaching the loader cartridge to the headpiece;
d. placing said at least two vials within the loading cartridge, such that one vial is placed within one open chamber, such that each exit port is in fluid connection with one of said vials; and
e. drawing the biological material of each vial into the barrel with which it is in fluid connection by withdrawing the plurality of plungers in the proximal direction of the device.

22. The method of claim 21, further comprising a cover securable to said distal end of said loader cartridge.

23. The method of claim 22, wherein said cover is secured to said loader cartridge via a hinge.

24. The method of claim 21, further comprising the steps of:
f. disconnecting said loader cartridge from said headpiece; and
g. connecting an applicator to the headpiece, said applicator having proximal and distal ends, comprising;
i. a mating structure at the proximal end of the applicator, the mating structure configured to be releasably engaged with the attachment mechanism on the headpiece;
ii. a dispensing structure disposed at the distal end of the applicator;
iii. a first and second fluid passageway in fluid communication with one of said exit ports;
iv. a first gas passageway in fluid communication with said gas exit port.

25. The method of claim 24, further comprising the steps of:
h. pressurizing the biological materials in each barrel by pressing on the plungers; and
i. expressing a pressurized fluid through said pressurized gas conduit.
